(19) United States
(12) Patent Application Publication
(10) Pub. No.: US 2016/0135999 A1
(43) Pub. Date: May 19, 2016

(54) Bandage System

(71) Applicant: Paul Mikoll, Colorado Springs, CO (US)

(72) Inventor: Paul Mikoll, Colorado Springs, CO (US)

(21) Appl. No.: 14/943,646

(22) Filed: Nov. 17, 2015

Related U.S. Application Data
(60) Provisional application No. 62/081,059, filed on Nov. 18, 2014.

(51) Int. Cl. A61F 13/00; A61F 13/02

(52) U.S. Cl. CPC A61F 13/00072; A61F 13/0269; A61F 13/00008

(57) ABSTRACT

A bandage system for dressing a wound. The bandage system includes an elongated bandage formed into a roll that can be cut to the size necessary to cover the user's wound. A separate strip of adhesive tabs is provided, wherein the adhesive tabs are removably secured to one another via perforated lines of connection. The adhesive tabs include a smooth outer surface and an underside surface having a pressure sensitive adhesive thereon. The adhesive tabs can be placed orthogonally to the bandage so as to be adhered to the user's skin and the bandage in order to securely position the bandage on the user. Additional adhesive tabs can be used to secure the bandage in the desired position. The elongated bandage and strip of adhesive tabs are preferably wound in a roll on a spool within a dispensing device, wherein the bandage and tabs can be withdrawn therefrom.

### CROSS REFERENCE TO RELATED APPLICATION

This application claims the benefit of U.S. Provisional Application No. 62/081,059 filed on Nov. 18, 2014. The above identified patent application is herein incorporated by reference in its entirety to provide continuity of disclosure.

### BACKGROUND OF THE INVENTION

1. Field of the Invention

The present invention relates to bandages. More specifically, the present invention provides a bandage system for dressing wounds comprising an elongated bandage that can be cut to a desired length so that the bandage can fully cover the user's wound. The bandage system further includes a strip of adhesive tabs removably secured to one another via perforated lines of connection. One or more of the adhesive tabs can be used to secure the bandage to the user's body.

It can often be difficult to dress a wound so as to completely cover and protect the site of injury. Large wounds, such as an elongated cut, can be difficult to dress using conventional bandages. Conventional bandages are a predetermined size, requiring the user to apply a plurality of bandages over a large cut or wound. Applying a plurality of small bandages to a wound can be time consuming, inconvenient and wasteful. Further, the relatively small size of the bandages may be insufficient to cover and protect the wound.

Additionally, conventional bandages include adhesives in predetermined locations, which may be undesirable where the user may not wish to have adhesive in some areas that may be sensitive or tender resulting from the injury. Further, it is preferable to use the least amount of adhesive possible when securing a bandage to a user so as to minimize the pain associated with removing the bandage and so as to avoid waste.

As an alternative to conventional bandages, people may apply gauze pads to a wound and then wrap the gauze pad with a fabric strap in order to secure the gauze pad in position. However, wrapping a limb with the fabric strap can be uncomfortable and the gauze pad may be bulky. Thus, a bandage system that the user can be cut to size for a particular wound and that can be easily secured with minimal adhesives is desired.

2. Description of the Prior Art

Devices have been disclosed in the prior art that relate to bandages. These include devices that have been patented and published in patent application publications. U.S. Pat. No. 4,530,353, U.S. Pat. No. 5,843,011, U.S. Pat. No. 5,480,377, U.S. Pat. No. 4,930,500, and U.S. Published Patent Application Number 2008/0039760 disclose adhesive bandages for covering wounds having adhesive portions and gauze or sponge pads for contacting the wound.

These devices, however, fail to disclose a bandage system comprising an elongated bandage composed of gauze or a similar substance that can be cut to size and disposed over a wound. Further, the prior art devices do not include a separate strip of adhesive tabs removably secured together via perforated lines of connection, wherein the adhesive tabs can be used to secure the bandage in position on the user's body.

In light of the devices disclosed in the prior art, it is submitted that the present invention substantially diverges in design elements from the prior art and consequently it is clear that there is a need in the art for an improvement to existing bandage devices. In this regard the instant invention substantially fulfills these needs.

### SUMMARY OF THE INVENTION

In view of the foregoing disadvantages inherent in the known types of bandages now present in the prior art, the present invention provides a new bandage wherein the same can be utilized for providing convenience for the user when dressing a large wound.

It is therefore an object of the present invention to provide a new and improved bandage device that has all of the advantages of the prior art and none of the disadvantages.

It is another object of the present invention to provide a bandage system providing a continuous strip of bandage that can be cut to size so as to fully cover a wound.

Another object of the present invention is to provide a bandage system comprising an elongated bandage and a plurality of adhesive tabs adapted for use in securing the elongated bandage to a user.

Yet another object of the present invention is to provide a bandage system comprising a bandage and a separate strip of adhesive tabs removably secured together via perforated lines of connection.

A further object of the present invention is to provide a bandage system comprising a dispensing device that stores a roll of bandage and a roll of adhesive tabs therein, wherein the roll of bandage and adhesive tabs can be withdrawn through slots of the housing.

Another object of the present invention is to provide a bandage system that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the bandage system of the present invention.

FIG. 2 shows a perspective view of the adhesive tabs of the present invention.

FIG. 3 shows a perspective view of the bandage of the present invention.

FIG. 4 shows a perspective view of a dispensing device.

FIG. 5 shows a cross-sectional view of the dispensing device.

### DETAILED DESCRIPTION OF THE INVENTION

Reference is made herein to the attached drawings. Like reference numerals are used throughout the drawings to depict like or similar elements of the bandage system. For the purposes of presenting a brief and clear description of the present invention, the preferred embodiment will be discussed as used for dressing a wound. The figures are intended for representative purposes only and should not be considered to be limiting in any respect.

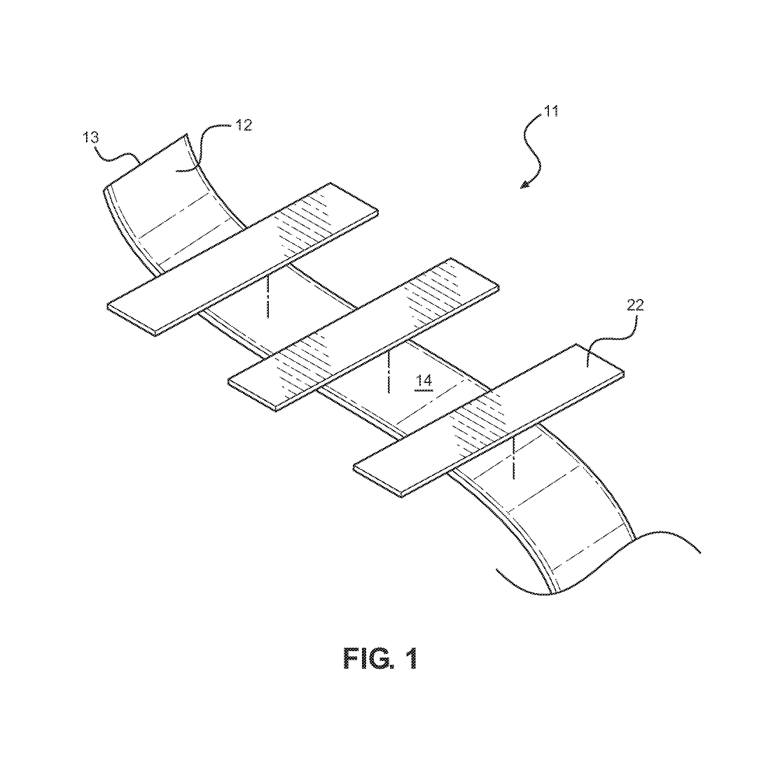

Referring now to FIG. 1, there is shown a perspective view of the bandage system of the present invention. The bandage system 11 of the present invention comprises an elongated strip of bandage 12 having a leading end 13. The bandage 12 is preferably planar and is rectangular in configuration. The bandage is composed of a soft, absorbent material, such as gauze or sponge-like material, and is adapted to be placed against a wound. The elongated bandage 12 can be cut to a desired length so that the user can select the exact length of elongated bandage 12 needed to cover the wound. The elongated bandage 12 further includes one or more perforations at fixed intervals along the length thereof to facilitate cutting the strip 12 to a desired size.

Adhesive tabs 22 are provided that are used to secure the elongated bandage 12 onto the user. The adhesive tabs 22 are preferably rectangular in configuration and include an adhesive disposed on the underside surface thereof. Preferably, the adhesive is a pressure sensitive adhesive. In operation, the user can position the elongated bandage 12 over a wound and can the secure the bandage 12 in position by attaching one or more adhesive tabs 22 over the upper surface 14 of the elongated bandage 12 in an orthogonal orientation. In this way, the adhesive tabs 22 adhere to both the elongated bandage 12 and to the user's skin in order to secure the elongated bandage 12 to the user.

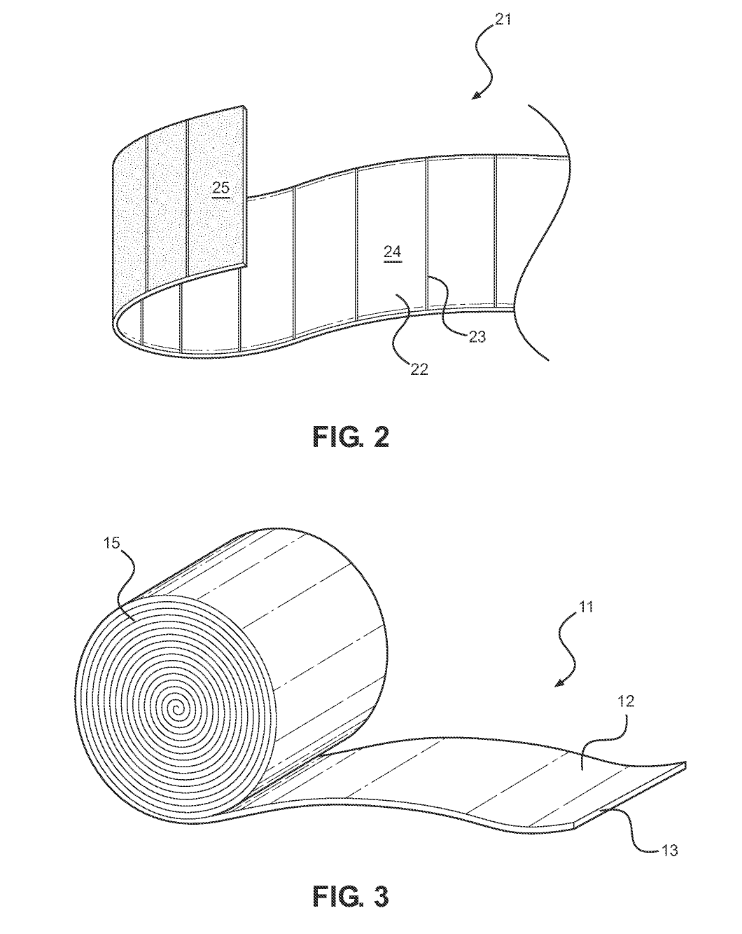

Referring now to FIG. 2, there is shown a perspective view of the adhesive tabs of the present invention. The adhesive tabs 22 are arranged on a strip 21. The strip 21 includes a plurality of adhesive tabs 22 removably secured together via perforated lines of connection 23 along the sides of the adhesive tabs 22. In this way, each adhesive tab 22 is the same size and dimension, and the adhesive tabs 22 can be easily separated from one another. The outer surface 24 of the adhesive tab 22 is preferably smooth and non-adhesive so that the adhesive tab 22 does not adhere to the user's clothing or other external objects. The underside surface 25 of the adhesive tab 22 includes an adhesive thereon. The adhesive preferably covers the entire underside surface 25 of the adhesive tab 22. The underside surface 25 may be removably covered by a backing that protects the adhesive until the time the adhesive tab 22 is desired to be used.

Referring now to FIG. 3, there is shown a perspective view of the bandage of the present invention. The bandage 12 is preferably stored in a roll 15 in order to provide a compact configuration for storage and transportation. The bandage 12 can be extended from the roll 15 so that the user can cut the bandage 12 to the desired length.

In operation, the bandage can be cut to a desired length so that a user can cover a wound with one continuous length of bandage. The user can separate one or more adhesive tabs from the roll of adhesive tabs via the perforations thereon. The adhesive tabs can be positioned orthogonally to the bandage so that the adhesive on the underside of the adhesive tab is secured to the user's skin and to the bandage in order to secure the bandage in position. The user can use additional adhesive tabs in order to firmly secure the bandage on the user. In this way, the present invention provides a convenient system for dressing a wound that allows the user to tailor the bandage to the size of the wound.

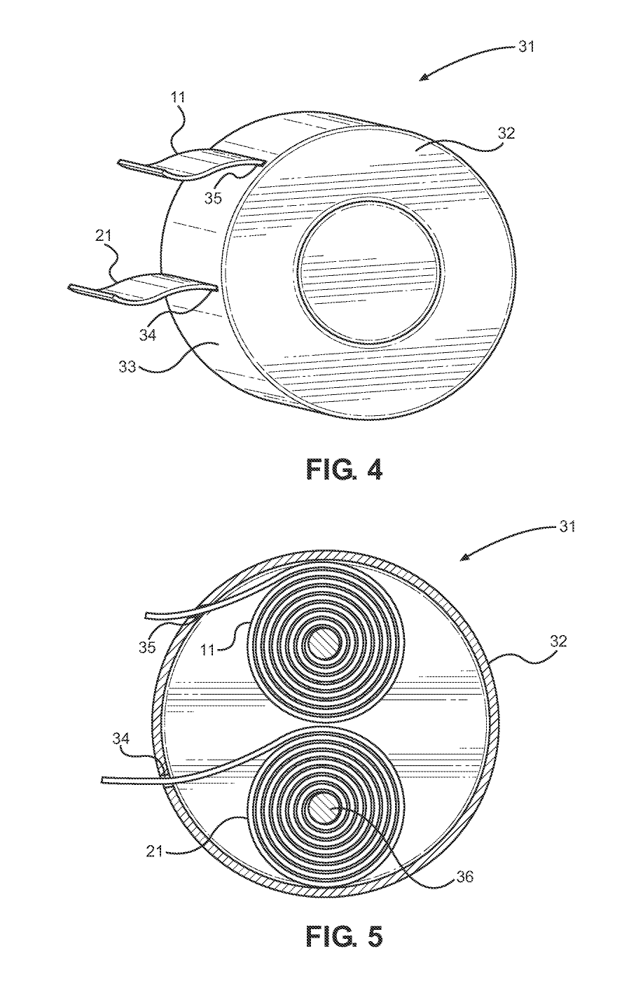

Referring now to FIGS. 4 and 5, there is shown a perspective view of a dispensing device and a side cross sectional view of the dispensing device, respectively. Preferably, the bandage 11 and strip of adhesive tabs 21 are stored in a rolled configuration within a dispensing device 31. In the illustrated embodiment, the dispensing device 31 comprises a housing 32 having a sidewall 33 and a hollow interior volume in which a roll of bandages and a rolled strip of adhesive tabs are positioned for storage. The housing 32 is shown as having a circular configuration, however, in alternate embodiments, the housing 32 may have other configurations. The roll of bandage 11 and the roll of adhesive tabs 21 are wound on rotatable spools 36 that allow the rolls to rotate thereon. The sidewall 33 of the housing 32 includes a first slot 34 and a second slot 35 through which the bandage 11 and adhesive strip 21 can extend for dispensing the same.

In operation, the user can pull the leading end of the bandage 11 so as to unwind a length of bandage from the spool 36 on which it is wound within the housing 32 of the dispensing device 31. The user can then cut or sever the bandage once the desired length of bandage 11 has been extended from the housing 32. Similarly, the user can pull the leading end of the strip of adhesive tabs 21 from the opening so as to unwind the tabs from the roll on the spool. The user can continue to pull or draw adhesive tabs 21 from the housing 32 until the desired number of tabs 21 has been exposed from the housing 32.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A bandage system for dressing a wound, comprising:
an elongated bandage adapted to be cut to a desired length;
a plurality of adhesive tabs removably secured to one another via perforated connections, wherein said plurality of adhesive tabs include a pressure sensitive adhesive disposed on an underside surface thereof adapted to be positioned on said elongated bandage and onto a user's skin in order to secure said elongated bandage to said user's skin.

2) The bandage system for dressing a wound of claim 1, wherein said elongated bandage is composed of gauze.

3) The bandage system for dressing a wound of claim 1, wherein said plurality of adhesive tabs comprise a smooth outer surface.

4) The bandage system for dressing a wound of claim 1, wherein said plurality of adhesive tabs are adapted to be arranged orthogonally with respect to said elongated bandage.

5) The bandage system for dressing a wound of claim 1, wherein said elongated bandage is configured in a roll.

6) A bandage system for dressing a wound, comprising:
a dispensing device comprising a housing having a hollow interior volume;
a first slot disposed on the housing, and a second slot disposed on the housing;
an elongated bandage wound in a roll on a first rotatable spool within the housing, wherein the elongated bandage extends outward through the first slot;
a strip of adhesive tabs wound in a roll on a second rotatable spool within the housing, wherein the strip of adhesive tabs extends outward through the second slot.
